(19) United States
(12) Patent Application Publication
(10) Pub. No.: US 2021/0230554 A1
(43) Pub. Date: Jul. 29, 2021

(54) GENERATION OF FUNCTIONAL BETA CELLS FROM HUMAN PLURIPOTENT STEM CELL-DERIVED ENDOCRINE PROGENITORS

(71) Applicant: Novo Nordisk A/S, Bagsvaerd (DK)

(72) Inventors: Nicolaj Stroeyer Christophersen, Virum (DK); Ulrik Doehn, Oelstykke (DK); Mattias Hansson, Malmoe (SE)

(21) Appl. No.: 17/271,635

(22) PCT Filed: Aug. 30, 2018
(86) PCT No.: PCT/EP2018/073342

§ 371 Date: Feb. 26, 2021

(51) Int. Cl. C12N 5/071; A61K 35/39; A61P 3/10

(52) U.S. Cl. CPC C12N 5/0676; A61K 35/39; A61P 3/10; C12N 2501/72; C12N 2501/15; C12N 2501/999; C12N 2501/01; C12N 2501/375; C12N 2501/845; C12N 2506/07; C12N 2501/91

(57) ABSTRACT

The present invention relates to method of generation of functional mature beta cells from human pluripotent stem cell-derived endocrine progenitors. The present invention also relates to functional mature beta cells produced by said methods and uses of said mature beta cells for treating diabetes.

### TECHNICAL FIELD

The present invention relates to methods of generating functional mature beta cells from human pluripotent stem cells derived endocrine progenitors.

### BACKGROUND

Islet cell transplantation has been used to treat type 1 diabetic patients showing superior glucose homeostasis compared with insulin therapy but this therapy is limited by organ donations. Human Pluripotent stem cells (hPSCs) such as human embryonic stem cells (hESCs) can proliferate infinitively and differentiate into many cell types, including beta cells (BCs) and may address the shortage of donor islets. Protocols to differentiate hPSC into definitive endoderm (DE), pancreatic endoderm (PE) cells and endocrine progenitors (EP) in vitro have been provided in WO2012/175633, WO2014/033322 and WO2015/028614 respectively. It is challenging to make glucose-responsive insulin-secreting BCs in vitro from hPSCs. Most protocols result in insulin-producing cells that fail to recapitulate the phenotype of BCs as they also co-express other hormones such as glucagon and are unresponsive to glucose stimulation.

Rezania, A. et al. “Reversal of diabetes with insulin-producing cells derived in vitro from human pluripotent stem cells” Nature Biotechnology 32, 1121-1133 (2014) and Pagliuca, F. W. et al. “Generation of Functional Human Pancreatic b Cells In Vitro” Cell 159(2), 428-439, Oct. 9, 2014, reported the in vitro differentiation of hESCs into insulin-secreting cells. Using static incubation studies, cells from both groups were sensitive to glucose stimulation showing approximately 2-fold increase in insulin output after glucose stimulation. This response varied however qualitatively and quantitatively from that of primary adult beta cells. As comparison, human islet stimulation index is reported to be two to ten or higher (Shapiro, J. A. M. et al. “Islet Transplantation in seven patients with type 1 diabetes mellitus using a glucocorticoid-free immunosuppressive regimen” New England Journal of Medicine 343, 230-238, July 27 (2000)). For example, WO2013163739 discloses methods and compositions for producing functional pancreatic beta cells, wherein endocrine progenitor cells are cultured with cAMP, Nicotinamide and TGF beta signaling pathway inhibitor. However, the pancreatic beta cells generated in vitro were not shown to be positive for insulin.

The reported stem cell-derived BCs also failed to display insulin response to glucose in a dynamic cell perfusion assay and are thus functionally immature relative to primary human BCs.

Efficient protocol for making functional mature BCs from hPSC-derived endocrine progenitors that can respond to glucose in a dynamic cell perfusion assay is not known. It is critical to improve current protocols to generate fully functional mature BCs for a more consistent cell product similar to human islets to obtain a predictable outcome following transplantation as well as for screening purposes in vitro.

### SUMMARY

The present invention relates to improved methods for generation of functional mature beta cells from human pluripotent stem cell-derived endocrine progenitors. The present invention also relates to glucose responsive fully differentiated beta-cells. The present invention further relates to functional mature beta cells obtainable by the methods of the present invention. The present invention further relates to medical use of said cells inter alia in the treatment of Type I diabetes. The present invention may also solve further problems that will be apparent from the disclosure of the exemplary embodiments.

### BRIEF DESCRIPTION OF DRAWINGS

FIG. 1 shows the screening approach where undifferentiated human embryonic stem cells (hESCs) were differentiated into Definitive Endoderm (DE) and reseeded in T75 flasks, where the cells were further differentiated into Pancreatic Endoderm (PE) and Endocrine Progenitor (EP). The Beta cells (BC) step 1 screen was started at the EP stage and continued for 4-7 days, and analysed by qICC monitoring and/or flow cytometry of NKX6.1+/INS+/GCG− cell number. BC step 2 screen was started at the end of BC step 1 screen and continued for 3-7 day period in 3D suspension cultures by dissociating to single cells at the end of BC step 1 and re-aggregation to clusters on orbital shaker at 50 rpm. Cells were analysed by static and/or dynamic GSIS, INS protein content, ICC, and qPCR.

FIG. 2 shows effect of compounds on the differentiation of Endocrine Progenitor co-expressing NKX2.2+/NKX6.1+(EP) into endocrine cells co-expressing INS+/NKX6.1+/GCG−. Flow cytometry (FC) measurement are taken at day 4 of BC step 1, i.e. EP cells culture 4 days in BC 1 step medium comprising DZNEP, Alk5i and heparin.

FIG. 3 shows additive effect of DAPT and dbcAMP when added to BC step 1 medium on the differentiation of endocrine progenitor cell into endocrine cells INS+/NKX6.1+ endocrine cell. (BC step 1 medium comprises DZNEP 1 uM, Alk5i 10 uM, Heparin 10 ug/ml and Nicotinamide 10 mM).

FIG. 4 shows timing studies to determine the optimal length of BC step 1 method based on mRNA expression of INS and GCG (BC step 1 medium comprises DZNEP 1 uM; Alk5i 10 uM; Heparin 10 ug/ml and Nicotinamide 10 mM).

FIG. 5 shows effect of selected compounds tested for a period of 7 days for induction of glucose responsive cells in static GSIS setup.

Functional beta cells are obtained from immature INS+/NKX6.1+ cells. (BC step 1 medium comprises: DZNEP 1 uM, Alk5i 10 uM, Heparin10 ug/ml and 10 mM Nicotinamide. BC step 2 medium comprises 12% KOSR).

FIG. 6.A shows presence of glucose and GLP1-responsive insulin secreting cells at day 3 of BC step 2 (BC step 1 medium comprises: DZNEP 1 uM, Alk5i 10 uM, Heparin 10 ug/ml and Nicotinamide 10 mM. BC step 2 medium comprises: 12% KOSR, 50 μM GABA, 10 μM Alk5i and 1 μM T3).

FIG. 6.B shows presence of glucose and GLP1-responsive insulin secreting cells at day 7 of BC step 2 (BC step 1 medium comprises DZNEP 1 uM, Alk5i 10 uM, Heparin 10 ug/ml and Nicotinamide 10 mM. BC step 2 medium comprises 12% KOSR, 50 μM GABA, 10 μM Alk5i and 1 μM T3).

FIG. 7. Perfusion analysis of the mature beta cells in response to elevated glucose and sulfonylurea tolbutamide.

Results show functionality of hESC-derived beta cells obtained at day 7 of BC step 2. The data demonstrated a significant additive effect of the sulfonylurea tolbutamid on insulin secretion (BC step 1 medium comprises DZNEP 1 uM, Alk5i 10 uM, Heparin 10 ug/ml and Nicotinamide 10 mM. BC step 2 medium comprises 12% KOSR, 50 μM GABA, 10 μM Alk5i, 1 μM T3).

FIG. 8 shows robustness of the protocol to differentiate EP into functional beta cells NKX6.1+/INS+ from independent pluripotent cell lines (BC step 1 medium comprises DZNEP 1 uM, Alk5i 10 uM, Heparin 10 ug/ml and Nicotinamide 10 mM. BC step 2 medium comprises 12% KOSR, 50 μM GABA, 10 μM Alk5i, 1 μM T3).

FIG. 9 shows Beta cell specific genes expressed in stem cell-derived beta cells at day 9 of BC step 2 (BC step 1 medium comprises DZNEP 1 uM, Alk5i 10 uM, Heparin 10 ug/ml and Nicotinamide 10 mM. BC step 2 medium comprises 12% KOSR, 50 μM GABA, 10 μM Alk5i and 1 μM T3).

FIG. 10 shows enrichment of key beta cell maturity genes after cell sorting for NKX6.1+/C− peptide double positive cells (BC step 1 medium comprises DZNEP 1 uM, Alk5i 10 uM, Heparin 10 ug/ml and Nicotinamide 10 mM. BC step 2 medium comprises 12% KOSR, 50 μM GABA, 10 μM Alk5i, 1 μM T3).

FIG. 11 shows rapid lowering of blood glucose and reversal of diabetes in diabetic mice transplanted with stem cell-derived beta cells from BC step 2.

(BC step 1 medium comprises DZNEP 1 uM, Alk5i 10 uM, Heparin 10 ug/ml and Nicotinamide 10 mM. BC step 2 medium comprises 12% KOSR, 50 μM GABA, 10 μM Alk5i and 1 μM T3).

FIG. 12 shows intraperitoneal glucose tolerance test (IPGTT) of transplanted stem cell derived beta cells from BC step 2.

(BC step 1 medium comprises DZNEP 1 uM, Alk5i 10 uM, Heparin 10 ug/ml and Nicotinamide 10 mM. BC step 2 medium comprises 12% KOSR, 50 μM GABA, 10 μM Alk5i and 1 μM T3)

FIG. 13 shows stem cell derived beta cells from BC step 2 protect against hyperglycemia post-streptozotocin treatment.

BC step 1 medium comprises DZNEP 1 uM, Alk5i 10 uM, Heparin 10 ug/ml and Nicotinamide 10 mM. BC step 2 medium comprises 12% KOSR, 50 μM GABA, 10 μM Alk5i and 1 μM T3)

FIG. 14 shows high levels of circulating human C-peptide in mice transplanted with stem cell derived beta cells from BC step 2.

(BC step 1 medium comprises DZNEP 1 uM, Alk5i 10 uM, Heparin 10 ug/ml and Nicotinamide 10 mM. BC step 2 medium comprises 12% KOSR, 50 μM GABA, 10 μM Alk5i and 1 μM T3)

FIG. 15 shows that stem cell derived endocrine progenitor cells can be differentiated in BC step 2 medium supplemented with 12% KOSR (FIG. 15A) or 2% B27 (FIG. 15B). (BC step 2 medium comprises RPMI1640 w Glutamax, 0.1% Pen/Strep, 12% KOSR or 2% B27, 50 μM GABA, 10 μM Alk5i, 1 μM T3 and 10 μM DZNEP)

FIG. 16 shows that stem cell derived endocrine progenitor cells can be differentiated in BC step 1 medium with or without Nicotinamide.

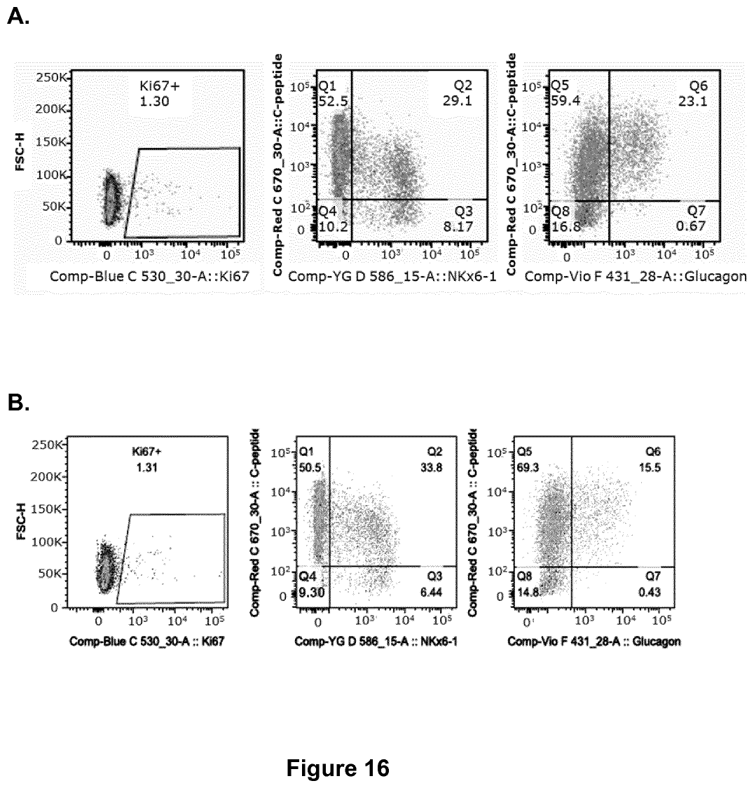

The use of BC step 1 medium with (FIG. 16A) or without (FIG. 16B) Nicotinamide does not affect the BC FACS phenotype. (BC step 1 medium comprises: RPMI1640 w Glutamax, 0.1% Pen/Strep, 12% KOSR, 10 μM Alk5i, 1 μM T3, 1 μM DZNEP, 10 μg/ml heparin, 25 μM DAPT, and +/−10 mM Nicotinamide).

### DESCRIPTION

The inventors of the present invention have performed extensive small-molecule screens and identified a novel and simple two-step method that generates functional mature Beta cells (BC) from the human pluripotent stem cell-derived endocrine progenitor stage.

The first step of the protocol (BC step 1) is for example a step of culturing EP cells in a medium (BC step 1 medium) for a sufficient period of time to induce high fraction of INS+ and NKX6.1+ double positive cells and only few GCG positive cells. The second step of the protocol (BC step 2) is for example a step of culturing cells obtained at BC step 1 in a medium (BC step 2 medium) for a sufficient period of time to allow to generate functional mature BC that respond strongly to repeated glucose challenges in vitro. Duration of BC step 1 is for example about 2 to 8 days, 3 to 7 days or 4 days, and duration of BC step 2 is for example about 2 to 14 days, 4 to 11 days or 11 days.

The inventors have shown the superiority of the mature beta cells obtained by the method of the present invention for glucose-stimulated insulin release dynamics measured by perfusion as compared to previous reports (Rezania, 2014; Paglucia, 2014 and review Johnson-J, 2016 Diabetologia). Importantly, the hPSC-derived BC cells respond to repeated glucose+/−Exendin4 challenges in a dynamic perfusion assay. The resulting functional mature BC also respond to increased glucose levels in vivo, 3 weeks after transplantation to the kidney capsule of non-diabetic mice. Dynamic insulin kinetics with rapid glucose response and low glucose shut-off are needed for successful safe stem cell therapy for Type 1 diabetes to prevent risk of glucose fluctuations, especially severe hypoglycemic events (Diabetologia. 2016 October; 59(10):2047-57. doi: 10.1007/s00125-016-4059-4. Epub 2016 Jul. 29)

Herein, it is provided a method for generating functional mature beta cells from human pluripotent stem cell-derived endocrine progenitors comprising the steps of:
(1) culturing said stem cell-derived endocrine progenitor cells in a cell culture medium comprising a serum replacement medium, histone methyltransferase EZH2 inhibitor, TGF-beta signaling pathway inhibitor and Heparin, to obtain INS+ and NKX6.1+ double positive immature beta cells, and
(2) culturing said INS+ and NKX6.1+ double positive immature beta cells of step (1) in a medium comprising a serum replacement medium (e.g. N2, B27 or KOSR) to obtain functional mature beta cells.

The inventors of the present invention have also found that gamma-Aminobutyric acid (GABA) administration in vivo following cell transplantation can potentially potentiate functional effect of transplanted BC.

In a preferred embodiment, the method for generating functional mature beta cells from human pluripotent stem cell-derived endocrine progenitors comprises the steps of:
(1) culturing said stem cell-derived endocrine progenitor cells in a cell culture medium comprising a serum replacement medium, histone methyltransferase EZH2 inhibitor, TGF-beta signaling pathway inhibitor and Heparin, to obtain INS+ and NKX6.1+ double positive immature beta cells, and
(2) culturing said INS+ and NKX6.1+ double positive immature beta cells of step (1) in a medium comprising a serum replacement medium (e.g. N2, B27 or KOSR) and GABA to obtain functional mature beta cells.

In a preferred embodiment, the histone methyltransferase EZH2 inhibitor is 3-Deazaneplanocin A (DZNEP). In a preferred embodiment, the TGF-beta signaling pathway inhibitor is Alk5iII.

In one embodiment, the serum replacement medium is selected from the group consisting of N2, KOSR and B27, preferentially 12% KOSR or 2% B27, more preferentially 12% KOSR.

In a preferred embodiment, the cell culture medium of step (1) comprises a serum replacement medium, histone methyltransferase EZH2 inhibitor, TGF-beta signaling pathway inhibitor, Heparin and Nicotinamide.

In a one embodiment, the cell culture medium of step (1) further comprises one or more additional agent(s) selected from the group comprising, gamma-secretase inhibitor, cAMP-elevating agent, thyroid hormone signaling pathway activator and combinations thereof.

In one embodiment, the additional agent of the cell culture medium of step (1) is a gamma-secretase inhibitor, preferentially is DAPT.

In one embodiment, the additional agent of the cell culture medium of step (1) is a cAMP-elevating agent, preferentially is dbcAMP.

In one embodiment, the additional agent of the cell culture medium of step (1) is a thyroid hormone signaling pathway activator, preferentially is T3.

In one embodiment, the additional agents of the cell culture medium of step (1) are gamma-secretase inhibitor and thyroid hormone signaling pathway activator, preferentially is DAPT and T3.

In one embodiment, the additional agent(s) of the cell culture medium of step (1) are gamma-secretase inhibitor and cAMP-elevating agent, preferentially is DAPT and dbcAMP.

In a preferred embodiment, in the cell culture medium of step (1) the cAMP-elevating agent is dbcAMP, the gamma-secretase inhibitor is DAPT, the thyroid hormone signaling pathway activator is T3.

In a preferred embodiment, the cell culture medium of step (1) comprises KOSR, DZNEP, Alk5iII, heparin, Nicotinamide, DAPT and T3.

In a preferred embodiment, the culture medium of step (2) comprises GABA.

In one embodiment, the culture medium of step (2) further comprises one or more additional agent(s) selected from group consisting of Nicotinamide, TGF-beta signaling pathway inhibitor, thyroid hormone signaling pathway activator, and/or histone methyltransferase EZH2 inhibitor.

In one embodiment, the additional agents of the culture medium of step (2) are TGF-beta signaling pathway inhibitor or a thyroid hormone signaling pathway activator or a histone methyltransferase EZH2 inhibitor, preferentially are respectively Alk5iII or T3 or DZNEP.

In one embodiment, the additional agents of the culture medium of step (2) are TGF-beta signaling pathway inhibitor and a thyroid hormone signaling pathway activator, preferentially are Alk5iII and T3.

In one embodiment, the additional agents of the culture medium of step (2) are TGF-beta signaling pathway inhibitor and a histone methyltransferase EZH2 inhibitor, preferentially are Alk5iII and DZNEP.

In one embodiment, the additional agents of the culture medium of step (2) are TGF-a thyroid hormone signaling pathway activator and a histone methyltransferase EZH2 inhibitor, preferentially are T3 and DZNEP.

In one embodiment, the additional agents of the culture medium of step (2) are thyroid hormone signaling pathway activator, a TGF-beta signaling pathway inhibitor and a histone methyltransferase EZH2 inhibitor, preferentially are respectively T3 and Alk5iII and DZNEP.

In a preferred embodiment, in the culture medium of step (2) the thyroid hormone signaling pathway activator is T3, TGF-beta signaling pathway inhibitor is Alk5iII and histone methyltransferase EZH2 inhibitor is DZNEP.

In a preferred embodiment, the culture medium of step (2) comprises KOSR, GABA, DZNEP, Alk5iII, T3 and/or Nicotinamide.

Also described herein are mature beta cells or composition comprising mature beta cells generated from the method according to the invention for use as a medicament or for use in treating Type I diabetes.

Also described herein are devices comprising mature beta cells or composition according to the invention.

The resulting fully functional BC population obtained according to the method of the invention can be used as an in vitro-based BC product to study human BC function, screening compounds for regulating insulin secretion, insulin protein processing, insulin secretion and—mechanism, GSIS studies, calcium influx signaling, autoimmune BC destruction, and BC trans differentiation.

Throughout this application terms method or protocol or process may be used interchangeably.

### Particular Embodiments

1. A method for generation of functional mature beta cells from human pluripotent stem cell-derived endocrine progenitors comprising the steps of

(1) culturing said stem cell-derived endocrine progenitor cells in a cell culture medium, comprising a serum replacement medium, histone methyltransferase EZH2 inhibitor, transforming growth factor beta (TGF)-beta signaling pathway inhibitor and Heparin, to obtain INS+ and NKX6.1+ double positive immature beta cells and

(2) culturing said INS+ and NKX6.1+ double positive immature beta cells of step (1) in a cell culture medium comprising a serum replacement medium, such as KOSR or B27, to obtain functional mature beta cells.

2. The method according to embodiment 1, wherein histone methyltransferase EZH2 inhibitor is 3-Deazaneplanocin A (DZNEP).
3. The method according to embodiment 2, wherein concentration of DZNEP is below 1 μM.
4. The method according to embodiment 2, wherein concentration of DZNEP is 1 μM.
5. The method according to embodiment 2, wherein concentration of DZNEP is in a range of 0.1-10 μM or 1-10 μM.
6. The method according to embodiment 2, wherein concentration of DZNEP is 10 μM.
7. The method according to embodiment 1, wherein transforming growth factor beta (TGF)-beta signaling pathway inhibitor is Alk5iII.
8. The method according to embodiment 7, wherein concentration of Alk5iII is below 1 μM.
9. The method according to embodiment 7, wherein concentration of Alk5iII is 1 μM.
10. The method according to embodiment 7, wherein concentration of Alk5iII is in a range of 0.1-10 μM or 1-10 μM.
11. The method according to embodiment 7, wherein concentration of Alk5iII is 10 μM.
12. The method according to embodiment 1, wherein concentration of Heparin is below 1 μg/ml.
13. The method according to embodiment 1, wherein concentration of Heparin is 1 μg/ml.
14. The method according to embodiment 1, wherein concentration of Heparin is in a range of 0.1-10 μg/ml or 1-10 μg/ml, preferentially the concentration of Heparin is 10 μg/ml.
15. The method according to any one of the preceding embodiments 1 to 14, wherein said cell culture medium is selected from the group comprising CMRL 1066, RPMI1640 medium and RPMI1640/Glutamax medium, preferentially RPMI1640/Glutamax medium.
16. The method according to any one of the preceding embodiments 1 to 15, wherein said serum replacement medium is selected from the group consisting of N2, KOSR and B27.
17. The method according to any one of the preceding embodiments 1 to 16, wherein the cell culture medium of step (1) further comprises Nicotinamide.
18. The method according to embodiment 17, wherein the concentration of Nicotinamide is below 1 mM.
19. The method according to embodiment 17, wherein the concentration of Nicotinamide is 1 mM.
20. The method according to embodiment 17, wherein the concentration of Nicotinamide is in a range of 0.1-10 mM or 1-10 mM, preferentially is 10 mM.
21. The method according to embodiment 17, wherein the cell culture medium of step (1) comprises DZNEP, Alk5iII, Heparin and Nicotinamide.
22. The method according to embodiment 21, wherein the cell culture medium of step (1) comprises 1 μM DZNEP, 10 μM Alk5iII, 10 μg/ml Heparin and 10 mM Nicotinamide.
23. The method according any one of the preceding embodiments 1 to 22, wherein the cell culture medium of step (1) further comprises one or more additional agent selected from a group consisting of gamma-secretase inhibitor, cAMP-elevating agent, thyroid hormone signaling pathway activator, and combinations thereof.
24. The method according to embodiment 23, wherein the additional agent is gamma-secretase inhibitor.
25. The method according to embodiment 24, wherein gamma-secretase inhibitor is N-[(3,5-Difluorophenyl)acetyl]-L-alanyl-2-phenyl]glycine-1,1-dimethylethyl ester (DAPT).
26. The method according to embodiment 25, wherein the concentration of DAPT is below 2.5 μM.
27. The method according to embodiment 25, wherein the concentration of DAPT is 2.5 μM.
28. The method according to embodiment 25, wherein the concentration of DAPT is in a range of 0.1-10 μM or 2.5-10 μM.
29. The method according to embodiment 25, wherein the concentration of DAPT is 5 μM.
30. The method according to embodiment 25, wherein the concentration of DAPT is 10 μM.
31. The method according to embodiment 23, wherein the additional agent is cAMP-elevating agent.
32. The method according to embodiment 31, wherein cAMP-elevating agent is Dibutyryl-cAMP (dbcAMP).
33. The method according to embodiment 32, wherein the concentration of dbcAMP is below 250 μM.
34. The method according to embodiment 32, wherein the concentration of dbcAMP is 250 μM.
35. The method according to embodiment 32, wherein the concentration of dbcAMP is in a range of 0.1-500 μM or 250-500 μM.
36. The method according to embodiment 32, wherein the concentration of dbcAMP is 500 μM.
37. The method according to embodiment 23, wherein the additional agent is thyroid hormone signaling pathway activator.
38. The method according to embodiment 37, wherein the thyroid hormone signaling pathway activator is T3.
39. The method according to embodiment 38, wherein the concentration of T3 is below 1 μM.
40. The method according to embodiment 38, wherein the concentration of T3 is 1 μM.
41. The method according to embodiment 38, wherein the concentration of T3 is in a range of 0.1-10 μM or 1-10 μM.
42. The method according to embodiment 38, wherein the concentration of T3 is 10 μM.
43. The method according to any one of the embodiments 1 to 23, wherein the cell culture medium of step (1) comprises DZNEP, Alk5iII, Heparin and Nicotinamide in combination with DAPT.
44. The method according to embodiment 43, wherein the cell culture medium of step (1) comprises 1 μM DZNEP, 10 μM Alk5iII, 10 μg/ml Heparin and 10 mM Nicotinamide in combination with 2.5 μM DAPT.
45. The method according to any one of embodiment 1 to 23, wherein the cell culture medium of step (1) comprises DZNEP, Alk5iII, Heparin and Nicotinamide in combination with dbcAMP.
46. The method according to embodiment 45, wherein the cell culture medium of step (1) comprises 1 μM DZNEP, 10 μM Alk5iII, 10 μg/ml Heparin and 10 mM Nicotinamide in combination with 250 μM dbcAMP.
47. The method according to embodiment 23, wherein the additional agents are gamma-secretase inhibitor and thyroid hormone signaling pathway activator.
48. The method according to embodiment 47, wherein the gamma-secretase inhibitor is DAPT and the thyroid hormone signaling pathway activator is T3.
49. The method according to embodiment 48, wherein the concentration of DAPT is 2.5 μM and the concentration of T3 is 1 μM.
50. The method according to embodiment 23, wherein the additional agents are gamma-secretase inhibitor and cAMP elevating agent.
51. The method according to embodiment 50, wherein the gamma-secretase inhibitor is DAPT and the cAMP elevating agent is dbcAMP.
52. The method according to embodiment 51, wherein the concentration of DAPT is 2.5 μM and concentration of dbcAMP is 250 μM.
53. The method according to any one of the preceding embodiments, wherein the stem cell-derived endocrine progenitor cells are cultured in step (1) for 1-4 days.
54. The method according to any one of the preceding embodiments, wherein the stem cell-derived endocrine progenitor cells are cultured in step (1) for 4 days.
55. The method according to any one of the preceding embodiments, wherein the stem cell-derived endocrine progenitor cells are cultured in step (1) for 4-7 days.
56. The method according to any one of the preceding embodiments, wherein 10-60% INS+ and NKX6.1+ double positive immature beta cells are obtained in step (1).
57. The method according to any one of the preceding embodiments, wherein 20-50% INS+ and NKX6.1+ double positive immature beta cells are obtained in step (1).
58. The method according to any one of the preceding embodiments, wherein 25-45% INS+ and NKX6.1+ double positive immature beta cells are obtained in step 1.
59. The method according to any one of the preceding embodiments, wherein 30-40% INS+ and NKX6.1+ double positive immature beta cells are obtained in step 1.
60. The method according to any one of the preceding embodiments, wherein the cell culture medium of step (2) further comprises GABA.
61. The method according to embodiment 60, wherein the concentration of GABA is in a range of 0.1-250 μM, or 50-250 μM.
62. The method according to embodiment 60, wherein the concentration of GABA is 50 μM.
63. The method according to embodiment 60, wherein the concentration of GABA is 250 μM.
64. The method according to any one of the preceding embodiments, wherein the cell culture medium of step (2) further comprises one or more additional agent(s) selected from the group consisting of Nicotinamide, TGF-beta signaling pathway inhibitor, thyroid hormone signaling pathway activator, and histone methyltransferase EZH2 inhibitor, to obtain functional mature beta cells.
65. The method according to embodiment 64, wherein additional agent is TGF-beta signaling pathway inhibitor.
66. The method according to embodiment 64 or 65, wherein TGF-beta signaling pathway inhibitor is Alk5iII.
67. The method according to any one of embodiments 64 to 66, wherein the concentration of Alk5iII is below 1 μM.
68. The method according to any one of embodiments 64 to 66, wherein the concentration of Alk5iII is 1 μM.
69. The method according to any one of embodiments 64 to 66, wherein the concentration of Alk5iII is in a range of 0.1-10 μM or 1-10 μM.
70. The method according to any one of embodiments 64 to 66, wherein the concentration of Alk5iII is 10 μM.
71. The method according to embodiment 64, wherein the additional agent is thyroid hormone signaling pathway activator.
72. The method according to embodiment 64 or 71, wherein the thyroid hormone signaling pathway activator is T3.
73. The method according to embodiment 72, wherein the concentration of T3 is below 1 μM.
74. The method according to embodiment 72, wherein the concentration of T3 is in a range of 0.1-10 μM or 1-10 μM.
75. The method according to embodiment 72, wherein the concentration of T3 is 1 μM.
76. The method according to embodiment 72, wherein the concentration of T3 is 10 μM.
77. The method according to embodiment 64, wherein additional agent is histone methyltransferase EZH2 inhibitor.
78. The method according to embodiment 64 or 77, wherein the histone methyltransferase EZH2 inhibitor is DZNEP
79. The method according to embodiment 78, wherein the concentration of DZNEP is below 1 μM.
80. The method according to embodiment 78, wherein the concentration of DZNEP is in a range of 0.1-10 μM or 1-10 μM.
81. The method according to embodiment 78, wherein the concentration of DZNep is 10 μM.
82. The method according to embodiment 64, wherein the additional agents are TGF-beta signaling pathway inhibitor, thyroid hormone signaling pathway activator and histone methyltransferase EZH2 inhibitor.
83. The method according to embodiment 82, wherein the thyroid hormone signaling pathway activator is T3, the TGF-beta signaling pathway inhibitor is Alk5iII and the histone methyltransferase EZH2 inhibitor is DZNEP.
84. The method according to embodiment 83, wherein the Alk5iII is in concentration of 10 μM, the T3 is in concentration of 1 μM and the DZNEP is in concentration of 1 μM.
85. The method according to embodiment 64, wherein the additional agents are TGF-beta signaling pathway inhibitor and thyroid hormone signaling pathway activator.
86. The method according to embodiment 85, wherein the TGF-beta signaling pathway inhibitor is Alk5iII and the thyroid hormone signaling pathway activator is T3.
87. The method according to embodiment 86, wherein the Alk5iII is in concentration of 10 μM and T3 is in concentration of 1 μM.
88. The method according to embodiment 64, wherein the additional agents are TGF-beta signaling pathway inhibitor and histone methyltransferase EZH2 inhibitor.
89. The method according to embodiment 88, wherein the TGF-beta signaling pathway inhibitor is Alk5iII and the histone methyltransferase EZH2 inhibitor is DZNEP.
90. The method according to embodiment 89, wherein the Alk5iII is in concentration of 10 μM and the DZNEP is in concentration of 1 μM.
91. The method according to any one of the preceding embodiments, wherein the immature beta cells obtained in step (1) are cultured in step (2) for 3-7 days.
92. The method according to any one of the preceding embodiments, wherein the immature beta cells obtained in step (1) are cultured in step (2) for 7-11 days.
93. The method according to any one of the preceding embodiments, wherein 10-60% functional mature beta cells are obtained in step 2.
94. The method according to any one of the preceding embodiments, wherein 20-50% functional mature beta cells are obtained in step 2.
95. The method according to any one of the preceding embodiments, wherein 25-45% functional mature beta cells are obtained in step 2.
96. The method according to any one of the preceding embodiments, wherein 30-40% functional mature beta cells are obtained in step 2.
97. Functional mature beta cells obtainable by the method according to any one of the preceding embodiments 1 to 96.
98. Functional mature beta cells obtained in embodiment 97, wherein said functional mature beta cells co-express MAFA, IAPP and G6PC2.
99. Mature beta cells generated from the method according to embodiments 1 to 96 for use as a medicament.
100. Mature beta cells generated from the method according to embodiments 1 to 96 for use in treating Type I diabetes.
101. Composition comprising mature beta cells generated from the method according to embodiments 1 to 96 for use in treating Type I diabetes.
102. Device comprising mature beta cells or composition according to embodiments 99 and 100.
103. The method according to embodiments 1-96, wherein the serum replacement medium is KOSR.
104. The method according to embodiment 103, wherein the KOSR is in a concentration between 5 and 20%, preferentially between 8 and 17%, more preferentially between 10 and 15%, even more preferentially is 8%, 10% or 12%.
105. The method according to embodiment 1, wherein serum replacement medium is B27.
106. The method according to embodiment 105, wherein B27 is in a concentration between 1 and 5%.
107. The method according to embodiment 105, wherein B27 is in a concentration of 2%.
108. The method according to embodiment 64, wherein said additional agent is Nicotinamide.
109. The method according to embodiment 64 or 108, wherein the concentration of Nicotinamide is below 1 mM.
110. The method according to embodiment 64 or 108, wherein the concentration of Nicotinamide is 1 mM.
111. The method according to embodiment 64 or 108, wherein the concentration of Nicotinamide is in a range of 0.1-10 mM or 1-10 mM.
112. The method according to embodiment 64 or 108, wherein the concentration of Nicotinamide is 10 mM.

In one embodiment, the cells obtainable by the method according to the invention are insulin producing cells, optionally together with cells differentiated towards glucagon, somatostatin, pancreatic polypeptide, and/or ghrelin producing cells. As used herein, “insulin producing cells” refers to cells that produce and store or secrete detectable amounts of insulin. “Insulin producing cells” can be individual cells or collections of cells.

In another embodiment, the cell population comprising pancreatic cells is obtained from a somatic cell population. In some aspects the somatic cell population has been induced to de-differentiate into an embryonic-like stem (ES, e.g., a pluripotent) cell. Such de-differentiated cells are also termed induced pluripotent stem cells (iPSC).

In another embodiment, the cell population comprising pancreatic cells is obtained from embryonic stem (ES, e.g., pluripotent) cells. In some aspects the cell population comprising pancreatic cells is pluripotent cells such as ES like-cells.

In another embodiment, the cell population comprising pancreatic cells is embryonic differentiated stem (ES or pluripotent) cells. Differentiation takes place in embryoid bodies and/or in monolayer cell cultures or a combination thereof.

In another embodiment, the cell population is a population of stem cells. In some aspects the cell population is a population of stem cells differentiated to the pancreatic endocrine lineage.

Stem cells are undifferentiated cells defined by their ability at the single cell level to both self-renew and differentiate to produce progeny cells, including self-renewing progenitors, non-renewing progenitors, and terminally differentiated cells. Stem cells are also characterized by their ability to differentiate in vitro into functional cells of various cell lineages from multiple germ layers (endoderm, mesoderm and ectoderm), as well as to give rise to tissues of multiple germ layers following transplantation and to contribute substantially to most, if not all, tissues following injection into blastocysts.

Stem cells are classified by their developmental potential as: (1) totipotent, meaning able to give rise to all embryonic and extraembryonic cell types; (2) pluripotent, meaning able to give rise to all embryonic cell types; (3) multi-potent, meaning able to give rise to a subset of cell lineages, but all within a particular tissue, organ, or physiological system (for example, hematopoietic stem cells (HSC) can produce progeny that include HSC (self-renewal), blood cell restricted oligopotent progenitors and all cell types and elements (e.g., platelets) that are normal components of the blood); (4) oligopotent, meaning able to give rise to a more restricted subset of cell lineages than multi-potent stem cells; and (5) unipotent, meaning able to give rise to a single cell lineage (e.g., spermatogenic stem cells).

As used herein “differentiate” or “differentiation” refers to a process where cells progress from an undifferentiated state to a differentiated state, from an immature state to a less immature state or from an immature state to a mature state. For example, early undifferentiated embryonic pancreatic cells are able to proliferate and express characteristics markers, like PDX1, NKX6.1, and PTF1a. Mature or differentiated pancreatic cells do not proliferate and do secrete high levels of pancreatic endocrine hormones or digestive enzymes. E.g., fully differentiated beta cells secrete insulin at high levels in response to glucose. Changes in cell interaction and maturation occur as cells lose markers of undifferentiated cells or gain markers of differentiated cells. Loss or gain of a single marker can indicate that a cell has “matured or fully differentiated.” The term “differentiation factor” refers to a compound added to pancreatic cells to enhance their differentiation to mature endocrine cells also containing insulin producing beta cells. Exemplary differentiation factors include hepatocyte growth factor, keratinocyte growth factor, exendin-4, basic fibroblast growth factor, insulin-like growth factor-1, nerve growth factor, epidermal growth factor platelet-derived growth factor, and glucagon-like peptide 1. In some aspects differentiation of the cells comprises culturing the cells in a medium comprising one or more differentiation factors.

As used herein, “human pluripotent stem cells” (hPSC) refers to cells that may be derived from any source and that are capable, under appropriate conditions, of producing human progeny of different cell types that are derivatives of all of the 3 germinal layers (endoderm, mesoderm, and ectoderm). hPSC may have the ability to form a teratoma in 8-12 week old SCID mice and/or the ability to form identifiable cells of all three germ layers in tissue culture. Included in the definition of human pluripotent stem cells are embryonic cells of various types including human blastocyst derived stem (hBS) cells in 30 literature often denoted as human embryonic stem (hES) cells, (see, e.g., Thomson et al. (1998), Heins et al. (2004), as well as induced pluripotent stem cells (see, e.g. Yu et al. (2007); Takahashi et al. (2007)). The various methods and other embodiments described herein may require or utilise hPSC from a variety of sources. For example, hPSC suitable for use may be obtained from developing embryos. Additionally or alternatively, suitable hPSC may be obtained from established cell lines and/or human induced pluripotent stem (hiPS) cells.

As used herein “hiPSC” refers to human induced pluripotent stem cells.

As used herein, the term “blastocyst-derived stem cell” is denoted BS cell, and the human form is termed “hBS cells”. In literature the cells are often referred to as embryonic stem cells, and more specifically human embryonic stem cells (hESC). The pluripotent stem cells used in the present invention can thus be embryonic stem cells prepared from blastocysts, as described in e.g. WO 03/055992 and WO 2007/042225, or be commercially available hBS cells or cell lines. However, it is further envisaged that any human pluripotent stem cell can be used in the present invention, including differentiated adult cells which are reprogrammed to pluripotent cells by e.g. the treating adult cells with certain transcription factors, such as OCT4, SOX2, NANOG, and LIN28 as disclosed in Yu, et al. (2007); Takahashi et al. (2007) and Yu et al. (2009).

As used herein, “serum replacement medium” refers to medium suitable to maintain cells in culture overtime. Such medium is known in the art, for example KOSR, B27 and N2. The medium concentration can be determined following the provider recommendation or can be adapted by the skilled person. For example, KOSR can be use according to the provider recommendation at a concentration of 20% (Thermofisher, KnockOut™ SR, Catalog number 10828010, 10828028). However, studies have shown that this medium can be efficiently used in a concentration in a range of 8% to 20% (Amit et al. 2000, “Clonally derived human embryonic stem cell lines maintain pluripotency and proliferative potential for prolonged periods of culture”; Neural Stem Cell Assays Editors(s): Navjot Kaur, Mohan C. Vemuri, First published: 30 Jan. 2015, page 190).

In one embodiment, the first step of the protocol (BC step 1) is a step of culturing EP cells during 2 to 8 days or 3 to 7 days. Preferentially, the first step of the protocol (BC step 1) is 4 days.

In one embodiment, the second step of the protocol (BC step 2) is a step of culturing cells obtained at BC step 1 during 3 to 14 days, 5 to 12 days or 7 to 11 days. Preferentially, the second step of the protocol (BC step 2) is 11 days.

All references, including publications, patent applications, and patents, cited herein are hereby incorporated by reference in their entirety and to the same extent as if each reference were individually and specifically indicated to be incorporated by reference and were set forth in its entirety herein (to the maximum extent permitted by law).

All headings and sub-headings are used herein for convenience only and should not be construed as limiting the invention in any way.

The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

While certain features of the invention have been illustrated and described herein, many modifications, substitutions, changes, and equivalents will now occur to those of ordinary skill in the art. It is, therefore, to be understood that the appended claims are intended to cover all such modifications and changes as fall within the true spirit of the invention.

### LIST OF ABBREVIATIONS

### AA: Activin A

BC: Beta cells
bFGF: basic fibroblast growth factor (FGF2)
D'Am: D'Amour protocol (Kroon et al., 2008)
DAPT: N-[(3,5-Difluorophenyl)acetyl]-L-alanyl-2-phenyl]glycine-1,1-dimethylethyl ester
DE: Definitive endoderm

### DZNEP: 3-Deazaneplanocin A

### EP: Endocrine Progenitor

FC: Flow cytometry
GABA: Gamma-Aminobutyric acid

### GCG: Glucagon

GSIS: Glucose stimulated insulin secretion
hESC: Human embryonic stem cells
hIPSC: Human induced pluripotent cells
hPSC: Human pluripotent stem cells
KOSR: Knockout serum replacement

### PE: Pancreatic Endoderm

RNA: Ribonucleic acid
PCR: Polymerase chain reaction
PS: Primitive streak

### EXAMPLES

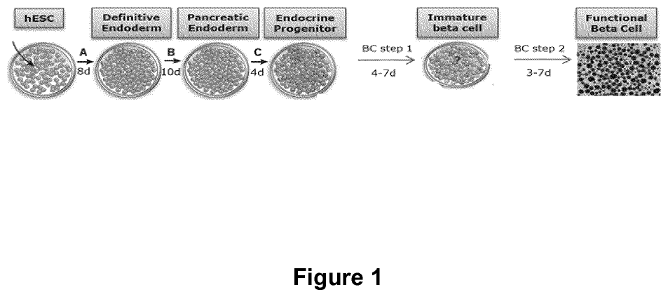

In general, the process of differentiating hPSCs to functional mature beta cells goes through various stages. An exemplary method for generating functional beta cells from hPSCs in vitro is outlined in FIG. 1.

### Example 1. Preparation of Endocrine Progenitor Cells

hESCs (SA121) were cultured in DEF media (Cellectis) supplemented with 30 ng/mL bFGF (Peprotech #100-18B) and 10 ng/mL noggin (Peprotech #120-10C).

For adherent cultures, the hESCs were differentiated into DE in T75 flasks using a Chir99021 and ActivinA based protocol in WO2012/175633. DE was trypsinized using Tryple Select (Invitrogen #12563-029) and reseeded as single cells in RPMI1640 supplemented with 100 ng/ml ActivinA (Peprotech #120-14E), 2% B27 (Invitrogen #17504-044) and 0.1% PEST (Gibco #15140) in T75 flasks at 200 K/cm². DE cells were allowed to attach and differentiated into PE using a LDN, AM508 based protocol in WO 2014/033322 followed by a four day EP protocol in WO2015/028614.

To produce large numbers of beta cells, a scalable suspension-based culture system was utilized by differentiating clusters of hESCs into DE in shaker flasks in Multitron Standard incubators (Infors) as suspension cultures (1 million/ml) at 70 RPM using a Chir99021 and ActivinA based protocol in WO2012/175633 without requirement of a reseeding step. DE cells were further differentiated into PE using a LDN, AM508 based protocol in WO 2014/033322 with the following slight modification: LDN is not added at PE day 4-10. Generation of PE was followed by a four day EP protocol WO2015/028614. Fully functional mature beta cells are generated following BC step 1 and BC step 2 method detailed below.

### Example 2. Screening for Factors that Induce INS+/NKX6.1+Co-Expression During BC Step 1

As a first step towards generating fully functional mature beta cells, we screened for factors to generate maximal numbers of immature INS+/NKX6.1+ cells (BC step 1 screen). BC step 1 screen was initiated at the EP stage (cf. endocrine progenitor cells co-expressing NGN3 and NKX2.2) using library of kinase inhibitors, epigenetic regulators, redox and bioactive lipids supplemented with some literature based compounds (in total 650 compounds of interests) added on top of a medium comprising RPMI1640+2% B27+10 mM Nicotinamide.

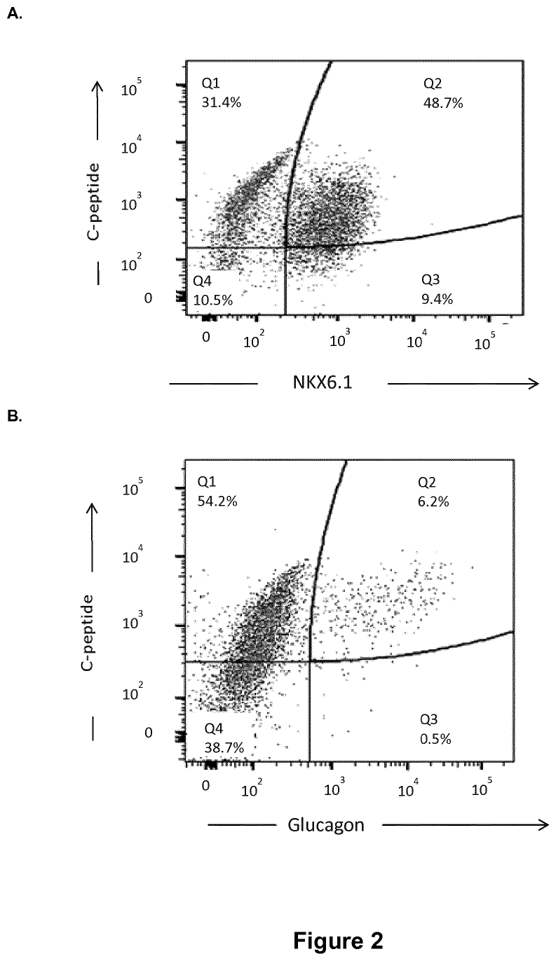

Compounds were screened for their ability to induce INS+, NKX6.1+ double positive immature BCs and few GCG positive cells within a 7 days period. Media change was performed daily. Cells were fixed at day 4 and day 7 of BC step 1 and analysed for INS NKX6.1 and GCG expression using flow cytometry (see Table 1 and FIG. 2). Briefly, differentiated endocrine cells were dispersed into single-cell suspension by incubation with TrypLE Express at 37° C. for 10 min. Differentiated endocrine cells were resuspended in 4% paraformaldehyde, washed in PBS followed by incubation with primary antibodies overnight and then secondary antibodies for 1 hour. The differentiated hPSCs co-expressed C-peptide+/NKX6.1+ with few cells expressing the α-cell hormone glucagon (FIG. 2). When quantified by flow cytometry, 48% of the cells co-expressed C-peptide+/NKX6-1 (FIG. 2), more than previously reported with stem cell-derived beta cells (Pagliuca et al., Cell. 2014 Oct. 9; 159(2):428-39. doi: 10.1016/j.cell.2014.09.040; Rezania et al., Nat Biotechnol. 2014 November; 32(11):1121-33. doi: 10.1038/nbt.3033. Epub 2014 Sep. 11).

TABLE 1
Flow cytometry analysis of BC step 1 method at BC step 1 day 7

| BC step 1 | RPMI1640 + 2% B27 + 1 uM DZNEP + 10 uM Alk5i + |
|---|---|
| medium | 10 ug/ml Heparin + 10 mM Nicotinamide. |
| INS+/NKX6.1+ | 20.8% |
| INS+/NKX6.1− | 19.8% |
| INS−/NKX6+ | 16.4% |
| INS/GCG | 12.3% |
| INS+/GCG− | 25.6% |
| INS−/GCG+ | 2% |

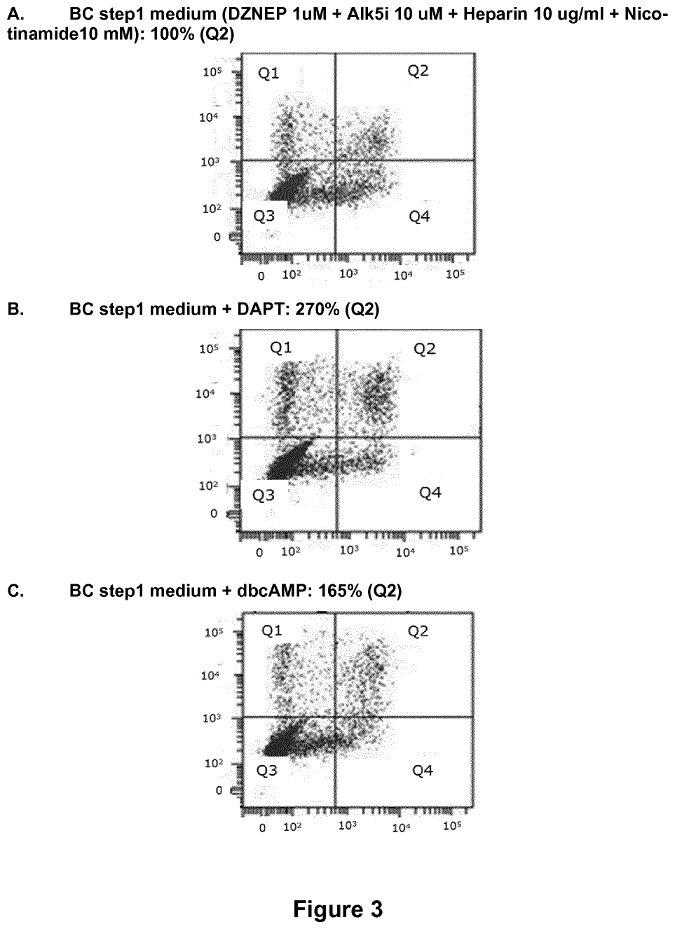

Hits compounds of Table 2 have been identified in a primary screen. Hits compounds of Table 2 were then combined individually and added on top of the BC step 1 medium (Results shows that BC step 1 medium comprising DZNEP, Alk5i, Heparin, Nicotinamide and DAPT or dbcAMP resulted in the highest number of INS+/NKX6.1+ cells FIG. 3).

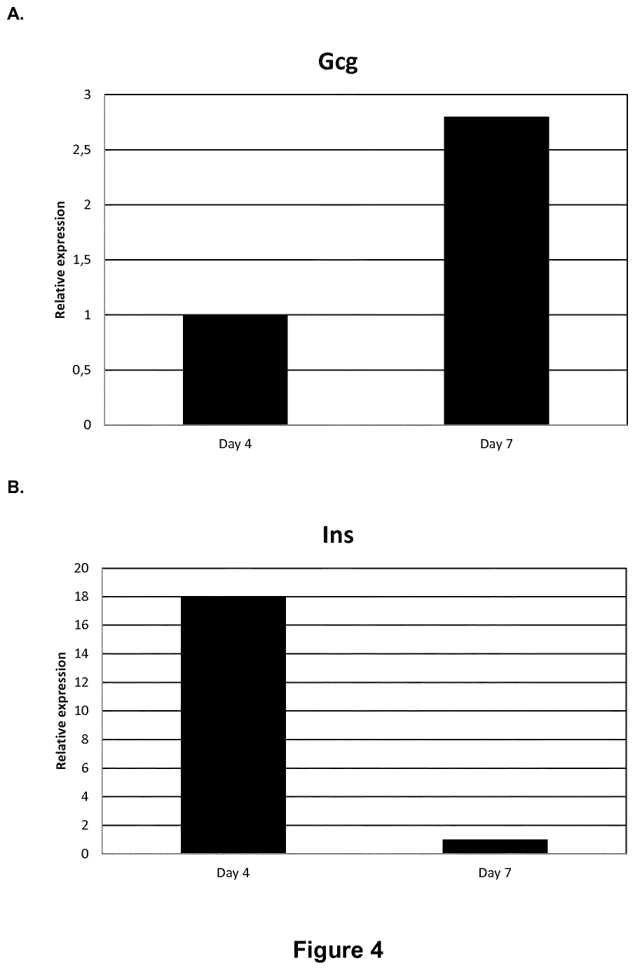

Timing of studies revealed that BC step 1 using BC step 1 medium comprising DZNEP, Alk5i, Heparin and Nicotinamide has an optimal length of 4-7 days based on mRNA expression of INS and GCG (see FIG. 4).

TABLE 2
Identified hit compounds for BC step 1 medium

| Compound name | Target | Structure | Concentration | |
|---|---|---|---|---|
| DAPT | Notch | 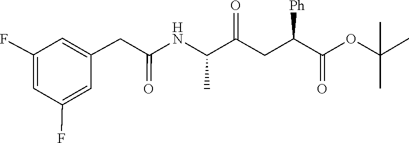 [SMILES: C[C@H](NC(=O)Cc1cc(F)cc(F)c1)C(=O)C[C@H](C(=O)OC(C)(C)C)c1ccccc1] | 2.5 | μM |
| ALK5iII | TGF-β RI Kinase | 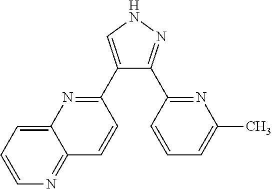 [SMILES: Cc1cccc(-c2n[nH]cc2-c2ccc3ncccc3n2)n1] | 1 μM-10 μM | |
| DZNEP | PRC complex? | 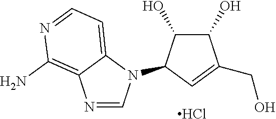 [SMILES: Nc1nccc2c1ncn2[C@@H]1C=C(CO)[C@@H](O)[C@H]1O ; Cl] | 1 μM-10 μM | |
| Heparin | | | 10 | μg/ml |
| dbcAMP | Increased cAMP levels | 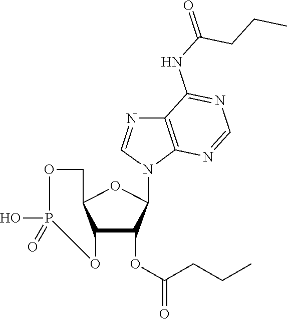 | 250 μM-500 μM | |
| Nicotinamide | | 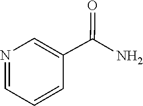 | 10 | mM |

| | | | |
|---|---|---|---|
| T3 | Thyroid receptor | 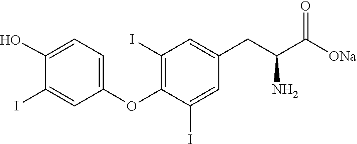 | 1 μM-10 μM |

### Example 3. Generation of Glucose Sensing Insulin Secreting Beta Cells from BC Step 1

The key functional feature of a fully functional mature beta cell is its ability to perform glucose stimulated insulin secretion (GSIS). We screened for factors in BC step 2 that could induce functional beta cells from the immature INS+/NKX6.1+ cells from BC step 1.

BC step 2 screen was performed in suspension cultures. For adherent cultures, cells in T75 flasks were trypsinized at the end of BC step 1 using Tryple Select, a recombinant cell-dissociation enzyme, ThermoFisher #12605036 and transferring cells into low attachment 9 cm petri dishes in suspension with RPMI1640 medium (Gibco #61870) containing 12% KOSR (ThermoFisher #10828028) and 0.1% PEST (Gibco #15140).

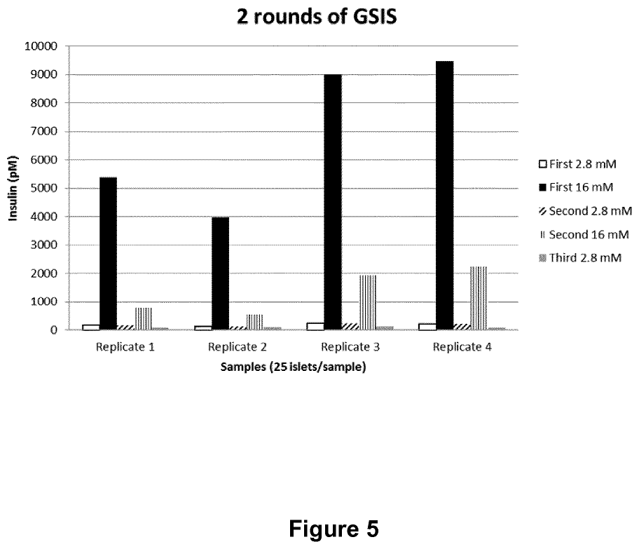

Effects of selected compounds see Table 3 were then tested for a 7 day period for induction of glucose-responsive cells in a static GSIS setup (see FIG. 5). Briefly, cell clusters were sampled and incubated overnight in 2.8 mM glucose media to remove residual insulin. Clusters were washed two times in Krebs buffer (1.26 M NaCl; 25 mM KCl; 250 mM NaHCO₃; 12 mM NaH₂PO₄; 12 mM MgCl₂; 25 mM CaCl₂), incubated in 2.8 mM Krebs buffer for 30 min, and supernatant collected. Then clusters were incubated in 16 mM glucose Krebs buffer for 30 min, and supernatant collected. This sequence was repeated. Finally, clusters were incubated in Krebs buffer containing 2.8 mM glucose for 30 min and then supernatant collected. Supernatant samples containing secreted insulin were processed using Human Insulin ELISA (Mercodia).

Hits identified in a primary screen were then combined individually and added on top of the BC 2 step medium (i.e. 12% KOSR medium) to generate the optimal 7-day BC step 2 medium, which comprises 50 μM GABA, 10 μM Alk5i, 1 μM T3.

TABLE 3
Identified hit compounds for BC step 2 medium

| Compound name | Target | Structure | Concentration | |
|---|---|---|---|---|
| T3 | Thyroid receptors | 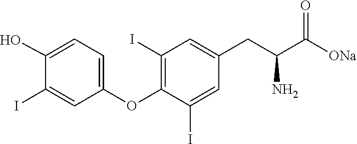 | 1 μM-10 μM | |
| ALK5iII | TGF-β RI Kinase | 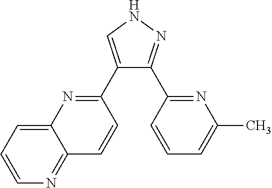 [SMILES: Cc1cccc(-c2n[nH]cc2-c2ccc3ncccc3n2)n1] | 1 μM-10 μM | |
| dbcAMP | cAMP | 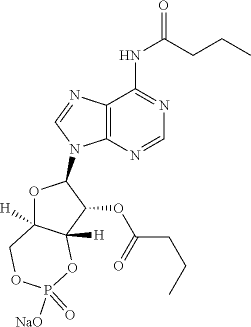 | 250 | μM |
| GABA | GABA receptors | 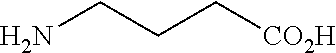 | 50 | μM |

### Example 4: Perfusion Assay to Assay Dynamic Human Insulin Secretion In Vitro

Mature beta cells are functionally defined by their rapid response to elevate glucose. Secretion of human insulin by beta cells at the end of BC step 2 method with BC step 2 medium comprising 50 μM GABA, 10 μM Alk5i, 1 μM T3 was measured as repeated responses to 20 mM glucose±1 μM exendin-4 a GLP1-receptor agonist or ±the anti-diabetic sulfonylurea compound Tolbutamide within a perfusion system.

Briefly, groups of 300 hand-picked, clusters of hESC- or hiPSC-derived cell clusters were suspended with beads (Bio-Rad #150-4124) in plastic chambers of Biorep PERFUSION SYSTEM (Biorep #PERI-4.2). Under temperature and CO₂-controlled conditions, the cells were perfused at 0.5 ml min−1 with a Krebs buffer. Prior to sample collection, cells were equilibrated under basal (2 mM glucose) conditions for 1 h. During perfusion cells were exposed to repeated challenges with 20 mM glucose±1 μM exendin-4 or ±100 μM Tolbutamide. At the end of perfusion, cells were exposed to cAMP-elevating agents (dbcAMP) on top of 20 mM glucose. Insulin secretion was measured by human insulin ELISA (Mercodia).

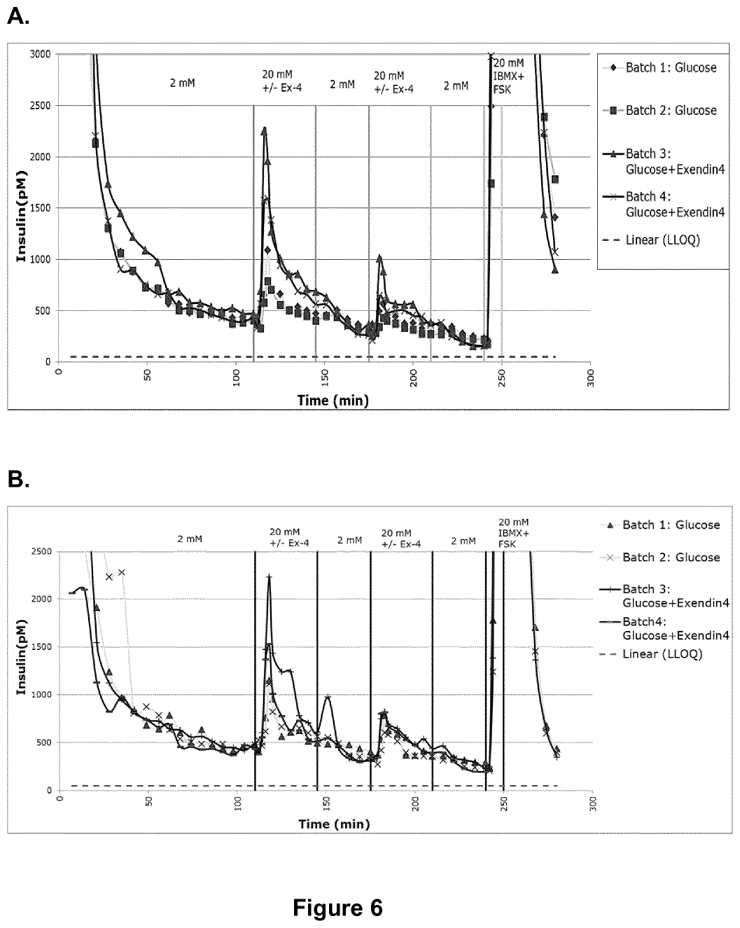

By perfusion analysis, our stem cell-derived beta cells exhibited rapid and robust release of insulin with a 1^{st} and 2^{nd} phase of insulin secretion that was highly synchronized with changes in glucose concentrations (see FIGS. 6 A and B). The GLP-1 analog exendin-4 increased the level of insulin secretion in the hPSC-derived beta cells. Importantly, presence of glucose and GLP1 responsive insulin secreting cells was observed for at least 4 days in vitro as measured at day 3 (FIG. 6A) and day 7 (FIG. 6B) of BC step 2.

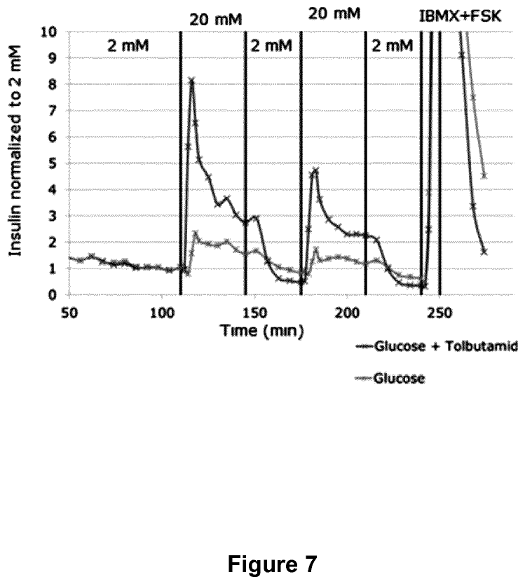

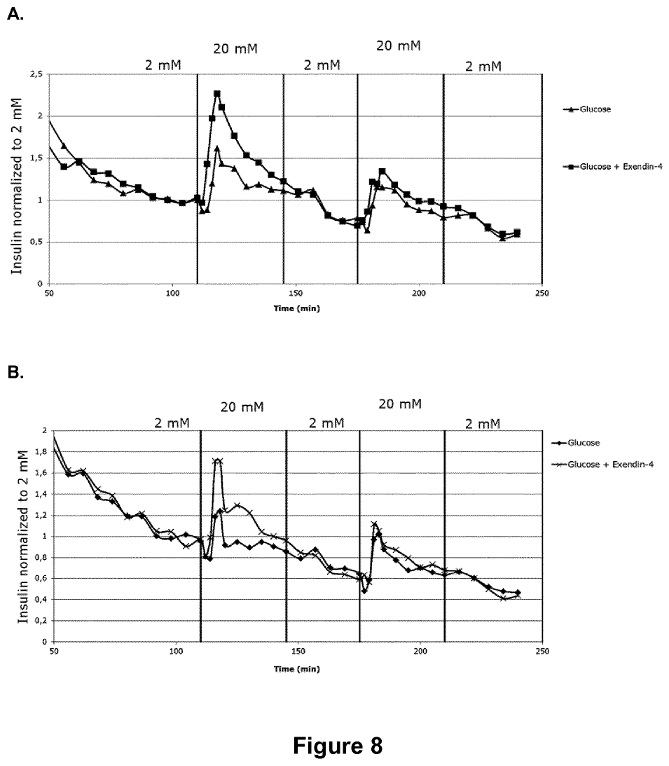

Another example of perfusion analysis of our stem cell-derived beta cells at day 7 of BC step 2 with BC step 2 medium comprising 50 μM GABA, 10 μM Alk5i, 1 μM T3 demonstrated a significant additive effect of the sulfonylurea tolbutamide on insulin secretion (FIG. 7). Robustness of the protocol is demonstrated by induction of functional beta cells from independent pluripotent cell lines (see FIG. 8). These data demonstrate collectively the superiority of the protocol for generating stem cell-derived beta cells that display glucose-stimulated insulin release dynamics measured by perfusion as compared to previous reports (Pagliuca et al., Cell. 2014 Oct. 9; 159(2):428-39. doi: 10.1016/j.cell.2014.09.040; Rezania et al., Nat Biotechnol. 2014 November; 32(11):1121-33. doi: 10.1038/nbt.3033. Epub 2014 Sep. 11; Johnson, J D. Diabetologia. 2016 October; 59(10):2047-57. doi: 10.1007/s00125-016-4059-4. Epub 2016 Jul. 29). Dynamic insulin kinetics with rapid glucose response and low glucose shut-off are needed for successful safe stem cell therapy for T1 diabetes to prevent risk of glucose fluctuations, especially severe hypoglycemic events.

### Example 5. Gene Expression Analysis Showed High Level of Similarities of Stem Cell-Derived Beta Cells to Human Islet Material

Differentiated cell clusters at day 7 of BC step 2 (BC step 2 medium comprising 50 μM GABA, 10 μM Alk5i, 1 μM T3) or human islets were collected and RNA was purified using the RNeasy kit from Qiagen (Cat No./ID: 74134). The quality was assessed using the RNA 6000 Nano Kit and the 2100 Bioanalyser instrument (Agilent). 100 ng RNA was subjected to an nCounter assay according to instructions from Nanostring Technology.

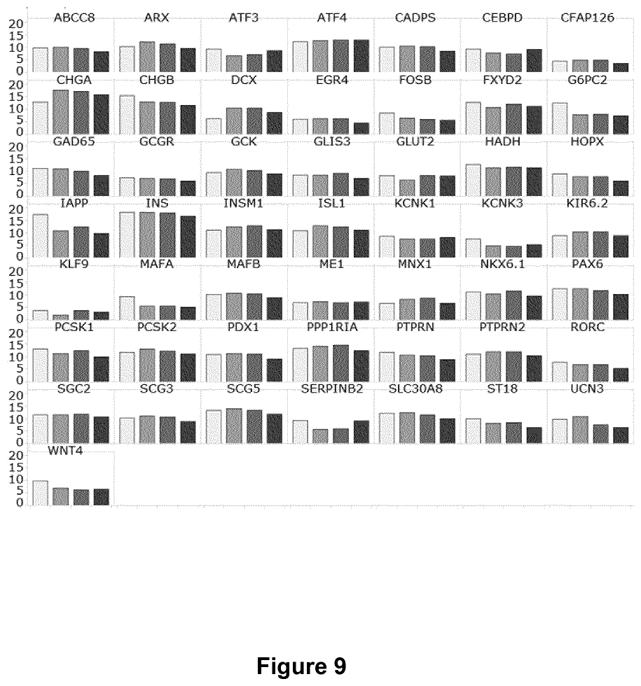

FIG. 9 shows the expression profile of beta cell associated genes from human islets and beta cells generated from hiPSC and two different hESC lines. Additional gene expression analysis of the specific stem cell-derived INS+/NKX6.1+ cells were performed by FACS cell sorting. Prior to sorting on the BD FACSAria Fusion™ instrument, cell clusters were dissociated and stained for the separation of live and dead cells using a near IR dye (Thermo Scientific). After fixation and permeabilisation the cells were stained using the intracellular markers NKX6.1 and C-peptide. RNA was purified using the RNeasy FFPE Kit (QIAGEN) and quality was assessed using the RNA 6000 Nano Kit and the 2100 Bianalyser instrument (Agilent).

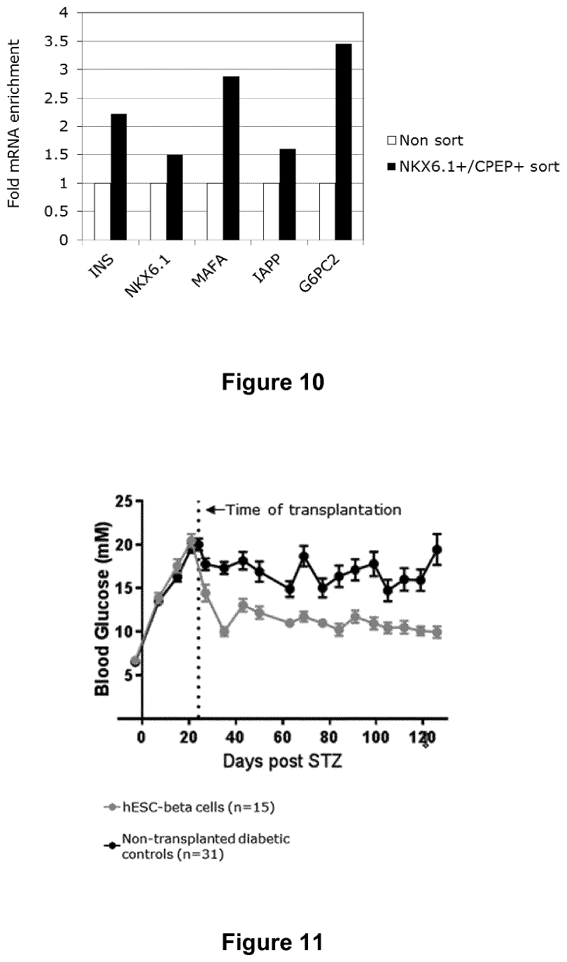

FIG. 10 shows enrichment of key beta cell maturity genes after cell sorting for NKX6.1/C-Peptide (CPEP) double positive cells. Nanostring data was normalized to the unsorted cell population.

The gene expression analysis showed that the stem cell-derived beta cells had close molecular resemblance to human islets.

### Example 6. Stem Cell-Derived Beta Cells from BC Step 2 Function after Transplantation

To evaluate functionality in vivo, stem cell-derived beta cells from day 3-10 of BC step 2 with BC step 2 medium comprising 50 μM GABA, 10 μM Alk5i, 1 μM T3 were transplanted into a streptozotocin-induced mouse model of diabetes. In short, diabetes is induced in immunocompromised scid-beige mice (Taconic) using Multiple Low Dose (5×70 mg/kg) Streptozotocin (STZ), the mice are fasted 4 h prior to STZ dosing. The mice are monitored over the following weeks with respect to blood glucose, body weight and HbA1c. Diabetes is considered when blood glucose is consistently above 16 mM.

In full anaesthesia and analgesia the diabetic mice are transplanted with 5×10⁶ human embryonic stem cell derived islet-like cells (unsorted population) under the kidney capsule. The kidney is exposed trough a small incision through skin and muscle of the left back side of the animal, a pouch between the parenchyma of the kidney and the capsule is created were the cell clusters are injected. The abdominal wall and the skin are closed and the mouse is allowed to recover.

The function of the cells is monitored over the coming weeks with respect to blood glucose, body weight, HbA1c and human C-peptide/insulin secretion. Our stem cell-derived beta cells resulted in rapid reversal of diabetes within the first two weeks after transplantation (FIG. 11), more rapidly than previous reports (Rezania, 2014). Importantly, all mice with less than 85% of bodyweight (BW) received daily injections with insulin, i.e. non-transplanted diabetic control group.

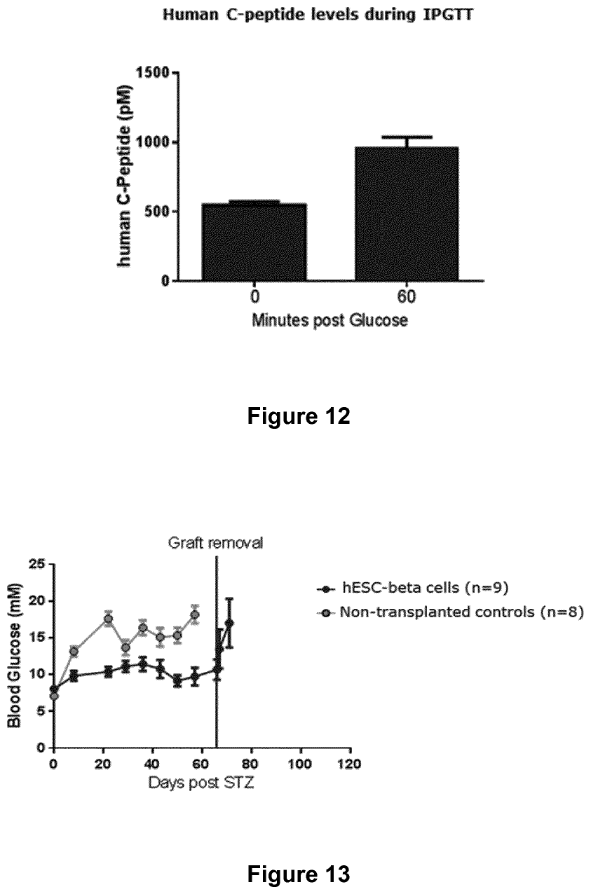

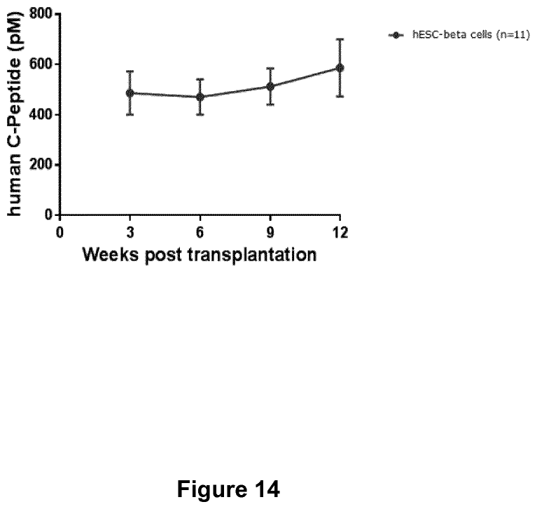

In vivo challenge of transplanted cells with glucose demonstrated in vivo functionality of our stem cell-derived beta cells with better glucose clearance than control mice and increased level of circulating human C-peptide within 60 min of glucose injection (FIG. 12). In another diabetes model, 5 million differentiated cells were transplanted to the kidney capsule of non-diabetic SCID/Beige mice. These mice were then treated with streptozotocin 8 weeks after transplantation. FIG. 13 demonstrates that the pre-transplanted mice were protected from hyperglycemia post-streptozotocin administration versus non-transplanted control mice, whereas removal of the graft resulted in rapid hyperglycemia in the mice (see FIG. 13). High levels of circulating human C-peptide was measured in all transplanted mice from the first data point and until end of study (see FIG. 14).

### Example 7. Effect of the Serum Replacement Medium KOSR and B27 of the BC Step 2 Medium

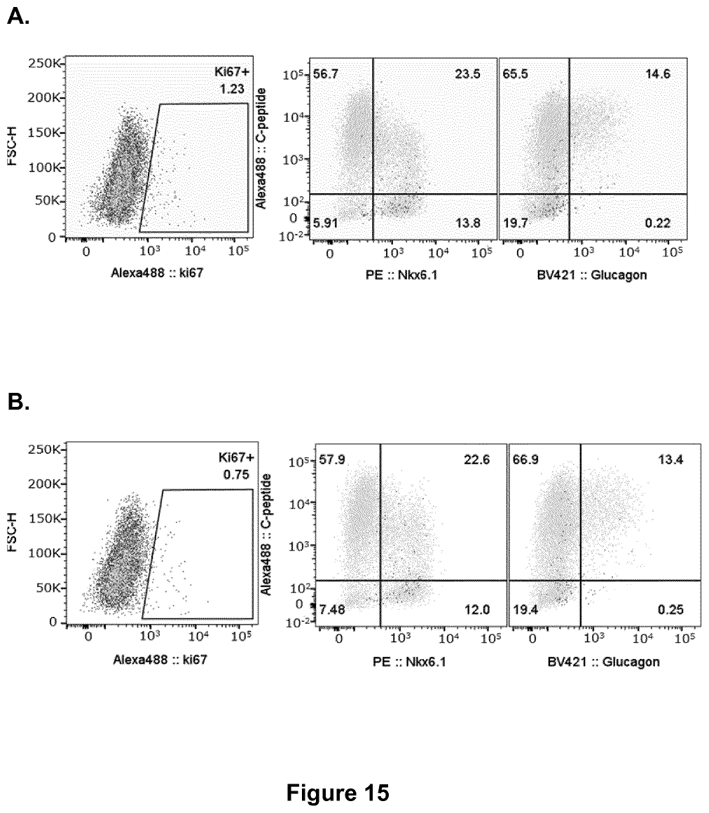

To evaluate the effect of KOSR of the BC step 2 medium, the 12% KOSR of the BC step 2 medium (FIG. 15A) was replaced by the serum replacement medium 2% B27 (FIG. 15B).

Cells were differentiated to endocrine progenitors and then subjected to BC step 1 medium for 4 days, consisting of RPMI1640 w Glutamax, 0.1% Pen/Strep, 12% KOSR, 10 μM Alk5i, 1 μM T3, 1 μM DZNEP, 10 μg/ml heparin, 25 μM DAPT, and 10 mM Nicotinamide.

In BC step 2, 12% KOSR was replaced with 2% B27 and thus the BC step 2 medium consisted of: RPMI1640 with Glutamax, 0.1% Pen/Strep, 12% KOSR or 2% B27, 50 μM GABA, 10 μM Alk5i, 1 μM T3 and 10 μM DZNEP

Results show that the BC FACS phenotype of the mature beta cells is not affected and that 12% KOSR can be replaced by 2% B27 in the BC 2 step medium (FIGS. 15 A. and B).

### Example 8. Effect of Nicotinamide of BC Step 1 Medium

To evaluate the effect of Nicotinamide, stem cell-derived endocrine progenitor cells were differentiated in BC step 1 medium with or without Nicotinamide, followed by a culturing step in BC step 2 medium.

Cell were differentiated to endocrine progenitors and then subjected to BC step 1 medium for 4 days (BC step 1 medium comprises RPMI1640 with Glutamax, 0.1% Pen/Strep, 12% KOSR, 10 μM Alk5i, 1 μM T3, 1 μM DZNEP, 10 μg/ml heparin, 25 μM DAPT, and with or without 10 mM Nicotinamide).

Cells were then subjected to BC step 2 cell culture medium for 2 days before analysing by FACS. BC step 2 medium consisted of: RPMI1640 with Glutamax, 0.1% Pen/Strep, 12% KOSR, 50 μM GABA, 10 μM Alk5i, 1 μM T3 and 10 μM DZNEP.

Results show that the BC FACS phenotype is not affected, which show that BC step 1 medium can be use with or without Nicotinamide (FIGS. 16 A. and B).

1. A method for generating functional mature beta cells from endocrine progenitor cells, comprising:
(1) culturing the endocrine progenitor cells in a cell culture medium comprising a first serum replacement medium, histone methyltransferase EZH2 inhibitor, TGF-beta signaling pathway inhibitor, and Heparin, to obtain INS+ and NKX6.1+ double positive immature beta cells and
(2) culturing the INS+ and NKX6.1+ double positive immature beta cells of step (1) in a cell culture medium comprising a second serum replacement medium, to obtain the functional mature beta cells.

2. The method according to claim 1, wherein the histone methyltransferase EZH2 inhibitor is 3-Deazaneplanocin A (DZNEP) and the TGF-beta signaling pathway inhibitor is Alk5iII.

3. The method according to claim 1, wherein the first serum replacement medium and the second serum replacement medium are individually selected from the group consisting of KOSR, B27, and N2.

4. The method according to claim 1, wherein the culture medium of step (1) further comprises Nicotinamide.

5. The method according to claim 1, wherein the culture medium of step (1) further comprises one or more additional agents selected from the group consisting of gamma-secretase inhibitor, cAMP-elevating agent, thyroid hormone signaling pathway activator, and combinations thereof.

6. The method according to claim 5, wherein the additional agents are gamma-secretase inhibitor and thyroid hormone signaling pathway activator, or are gamma-secretase inhibitor and cAMP-elevating agent.

7. The method according to claim 5, wherein the gamma-secretase inhibitor is DAPT, the cAMP-elevating agent is dbcAMP, and the thyroid hormone signaling pathway activator is T3.

8. The method according to claim 1, wherein the culture medium of step (2) further comprises GABA.

9. The method according to claim 1, wherein the culture medium of step (2) further comprises one or more additional agents selected from group consisting of Nicotinamide, TGF-beta signaling pathway inhibitor, thyroid hormone signaling pathway activator, and histone methyltransferase EZH2 inhibitor.

10. The method according to claim 9, wherein the additional agents are TGF-beta signaling pathway inhibitor and thyroid hormone signaling pathway activator.

11. The method according to claim 9, wherein the TGF-beta signaling pathway inhibitor is Alk5iII, the thyroid hormone signaling pathway activator is T3 and, the histone methyltransferase EZH2 inhibitor is DZNEP.

12. (canceled)

13. An in vitro composition comprising the mature beta cells according to claim 1.

14. A method for treating Type I diabetes, comprising administering the mature beta cells according to claim 1 to a person in need thereof.

15. A device comprising mature beta cells generated from the method according to claim 1.

16. The method according to claim 9, wherein the additional agents are TGF-beta signaling pathway inhibitor, thyroid hormone signaling pathway activator, and histone methyltransferase EZH2 inhibitor.
